(19) United States
(12) Patent Application Publication
(10) Pub. No.: US 2012/0149980 A1
(43) Pub. Date: Jun. 14, 2012

(54) VIDEO CRICOTHYROTOMY CAMERA AND CANNULA SYSTEM

(75) Inventor: John A. Pacey, Vancouver BC (CA)

(73) Assignee: VERATHON MEDICAL (CANADA) ULC, Burnaby (CA)

(21) Appl. No.: 13/313,822

(22) Filed: Dec. 7, 2011

Related U.S. Application Data
(60) Provisional application No. 61/421,080, filed on Dec. 8, 2010.

(51) Int. Cl. A61M 16/04; A61B 1/04

(52) U.S. Cl. 600/109

(57) ABSTRACT

A substantially rigid cannula having a channel configured to sequentially house a slidably removable video camera and to provide a ventilating airway upon sealable engagement of a ventilation extension with the channel after the camera is removed. The camera is connected with a monitor to guide the placement of the cannula and its placement within the tracheal lumen. Upon engagement with the channel and a ventilating source, a breathable airway is established through the cannula.

### PRIORITY CLAIM

This application claims priority to U.S. Provisional Patent Application Ser. No. 61/421,080 filed Dec. 8, 2010 and is incorporated by reference as if fully set forth herein.

### FIELD OF THE INVENTION

Disclosure herein is generally directed to the field of cricothyrotomy related devices, systems, and methods.

### BACKGROUND OF THE INVENTION

The application of video to airway tasks has been primarily via the flexible fiber optic method and the check of tube placements has been carried out via tracheostomy, endotracheal, and double lumen lung isolation tubes. Barthel and Shikani have described a flexible scope of the type. In addition Pacey describes a video laryngoscope that is used to view the pharynx during intubation such that airway video applications greatly contribute to difficult airway management.

None have applied video to a product designed to perform cricothyrotomy. The primary problems of Cricothyrotomy are based on the difficulty of obtaining a certain fix on or targeting of the cricothyroid membrane and then being certain of the placement of a ventilating catheter within the tracheal lumen. Methods to find the membrane have been clinical, by locating the thyroid cartilage and focusing on the lower margin of this structure or by using ultrasound devices.

The problems inherent in established approaches to cricothyrotomy include:
1. Failure to identify the midline
2. Failure to identify the level of the cricothyroid membrane
3. Inadequate incision
4. Bleeding
5. Damage to the trachea
6. Failure to secure a seal so that the air is forced into the subcutaneous space around the neck.

Furthermore, the confirmation of the lumen may be done by injection of local anesthetic and aspiration of air from the lumen but this does not affirm the final position of the ventilation catheter and therefore may lead to a degree of uncertainty that can result in poorly directed ventilation efforts. The complications include surgical emphysema, death due to inadequate ventilation, and perforation of the trachea. Fortunately, as described below, there are substantially new systems, devices, and methods to overcome these problems.

### BRIEF DESCRIPTION OF THE DRAWINGS

Preferred and alternative examples of the present invention are described in detail below with reference to the following drawings:

FIG. 1 schematically depicts a video-based cricothyrotomy ventilation system;

FIG. 2 schematically depicts the video camera removed from the rigid cannula;

FIG. 3 schematically depicts an anterior view of the rigid cannula;

FIG. 4 schematically depicts a side cross-sectional view of the rigid cannula;

FIG. 5 schematically depicts a perspective view of the insertable and removable video camera;

FIG. 6 schematically depicts a side view of the ventilation adapter inserted into the handle portion of the rigid cannula;

FIG. 7 schematically depicts a perspective view of the attachment of the ventilation tubes to the ventilation adapter;

FIGS. 8-10 depicts the anatomical location and preparation of the cricothyroid membrane for receiving entry of the rigid cannula;

FIG. 11 schematically depicts the camera-loaded cannula's blade region into the tracheal lumen with the video camera viewing inferiorly towards the lungs of the patient;

FIG. 12 schematically depicts the camera-loaded cannula's handle region lodged from the patient's neck in which the camera provides an interior view of the tracheal lumen presented on the monitor showing a substantially centered blade region as indicated by the tracheal lumen appearing in the middle of the monitor image;

FIG. 13 schematically depicts a perspective view of the attached ventilator tubes pneumatically in communication with the ventilator adapter snugly held within the cannula's handle region that protrudes from the patient's neck;

FIG. 14 schematically depicts a method to of using the video-based cricothyrotomy ventilation system to secure a breathing communication with the patient's airway.

### DETAILED DESCRIPTION OF THE INVENTION

Embodiments described below relate to a substantially rigid cannula having an inflatable cuff and a channel configured to sequentially house a slidably removable video camera and to provide a ventilating airway upon sealable engagement of a ventilation extension with the channel after the camera is removed. The camera is connected with a monitor to guide the placement of the cannula and its subsequent anchoring via an inflatable cuff within the tracheal lumen. That is, the channel serves both to temporarily house the video camera to provide the images for catheter positioning and anchoring, and also to serve as a ventilating catheter after the cameras removal from the channel. Upon sealable engagement of the ventilation extension with the channel and a ventilating source, a breathable airway is established through the cannula's channel.

The system relates to placement of cannula to patients who have undergone some emergency surgical preparation, that is, patients that have received a cricothyroid membrane incision. A long video baton having a light source and a heated lens temporarily is housed within the channel. The channel provides an open airway conduit within the cannula placed into and through the cricothyroid membrane incision upon the video camera removal to transition to ventilation from the video camera loaded device.

The primary ventilation catheter may be designed from a video laryngoscope sterile component which has a hollow ventilation channel with no front lens cover. With this part the video baton can be used to determine if the cannula is in the correct place to begin ventilation. The ventilation adapter can then be placed to provide a connection from the ventilator to the ventilation cannula and allow positive pressure ventilation to begin. The provision of a cuff on the cannula will allow closure of the substantial expected air leak around the catheter and permit the development of positive pressure in the airways such that ventilation will be achieved.

Video may be applied through the lumen of the cricothyrotomy cannula after intubation or during the act of intubation so that the access to the trachea at the level of the cricothyrotomy catheter will be obtained. The configurement of the systems described below allows those practitioners unfamiliar with cricothyrotomy to confidently execute this critical procedure with simplified training and basic surgical skill level. Thus the video provides a needed assist for practitioners to accurately locate the ventilation cannula with video prior to ventilation.

In other embodiments configurations allow for video guided placement of a ventilating airway to those very obese patients having a short neck. In these circumstances some extraordinary measures are required and therefore some education to the practitioner is necessary. Whenever difficulty is anticipated one may mark the site and confirm with injection and air aspiration or even leave in a tracer wire to mark the appropriate site of intervention.

The described system may be deployed with certainty any time the anatomy is correctly identified. The tip of the device may be semi-sharp to more easily allow penetration to the tracheal level whenever the incision in the cricothyroid membrane is not wide enough to accommodate the blade shaft portion of the rigid cannula. This allows a tighter incision to shaft fit thereby minimizing ventilation leakage around the cannula to skin contact region. The rigid cannula's tip, though configured to penetrate an incision previously placed in the cricothyroid membrane, is rounded enough not to stab through the more distal trachea wall upon the tips penetration via the incision into the tracheal lumen from the proximal trachea wall. The video may be held back a few mms during insertion to avoid lens contamination and then placed fully forward when the airway is entered. The safety thus obtained is useful and the blunt nature of the device tends to reduce the hazards to tracheal damage. The ventilating cannula may include a small 6 mm internal diameter that narrows over a short distance to allow it to move large to minute volumes through the camera channel between the patient and the ventilating source. In alternate embodiments the camera may be mounted to a baton that has a flexible and controllable tube that houses the camera to allow extending the camera beyond the distal cannula aperture to acquire images to facilitate aiming of the cannula and its placement within the tracheal lumen.

Embodiments include an intubation system for the trachea having a cannula with a hollow center channel which permits ventilation and passage of a video baton. Alternate embodiments provide for the intubation system and ventilating cannula to be angulated at a 5-120 degree angle to permit airway entry, with a preferred range being 60-90 degrees.

The cannula is further configured with a means to secure the intubation system into the airway and has a lumen of varying cross section which has a flow delivery capacity equal to or better than a 7.0 mm endotracheal tube and has ability to lock in place the camera and also the ventilator adapter channel. Upon removal of the video camera, the ventilator connector or extension provides or converts the video channel to a ventilating channel and has means to lock this in place.

The ventilating adapter or extension is configured to attach to a ventilator hose apparatus with a 15 mm standard type of connector and the other end to insert into the cannula. Press fitting to provide a sealing connection or other locking features to allow a secure pneumatic ventilation during the ventilation phase.

Other embodiments provide for the ventilating and camera housing channel to provide a seal conformal to the outer cannula channel so that positive pressure may be transmitted to the airway for ventilation. The seal is optional and is provided to facilitate the frictional forces conveyed by the press-fitting between the exterior surfaces of the ventilating adapter with the interior surfaces of the handle region's lumen of the substantially rigid cannula.

The cannula's distal may be configured to have a balloon for inflation, such as a pilot balloon, an inflation channel, and a valve to retain the inflation pressure. The balloon, pilot balloon, and the inflation channel is optional and provides both an assist to minimizing air exchange leakages around the contact points of the cannula's blade region with the cricothyroid membrane incision. Additionally, the balloon, the pilot balloon, and the inflation channel provides a more secure anchoring as through frictional forces conveyed by the expanded balloon's pressing contact with the tracheal walls.

The video or visualizing means may be any type of fiber optic or video insert that will allow image acquisition and transmission to a monitor for external viewing by care team members. The video camera also provides for an appropriate lighting means and for the capability to flexibly adapt to the cannula's channel.

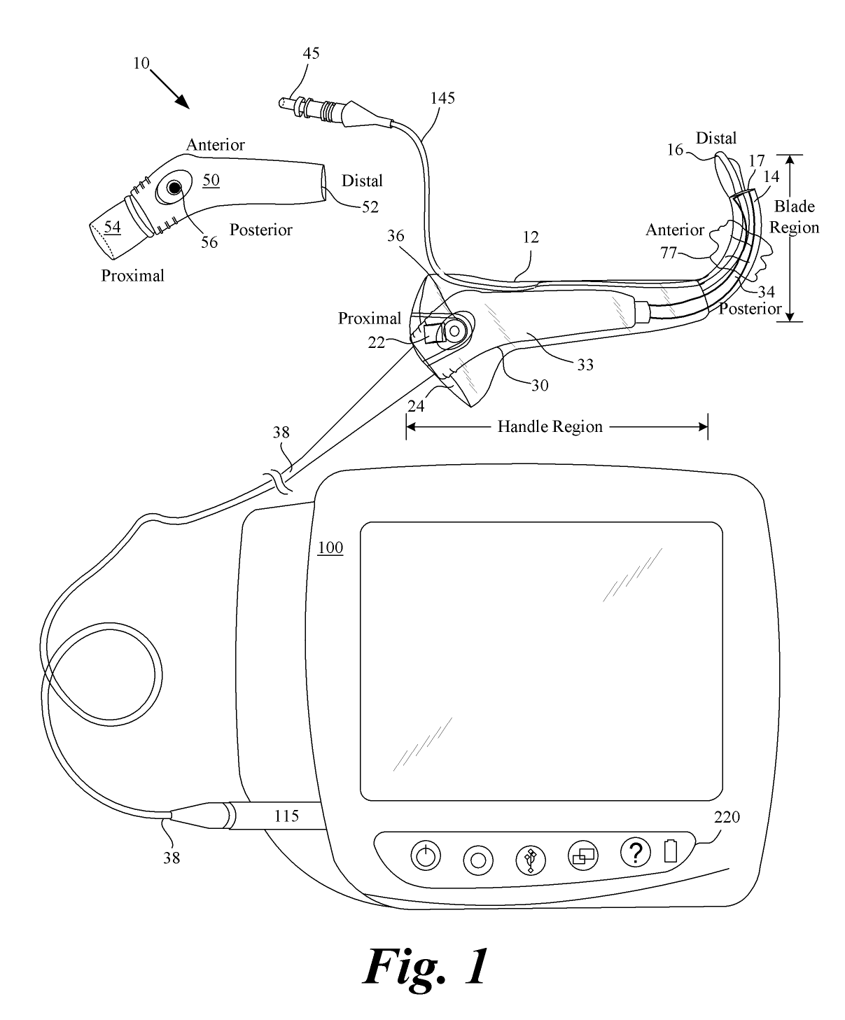

FIGS. 1-14 illustrate and are further described below for the various particular embodiments of the invention referenced above.

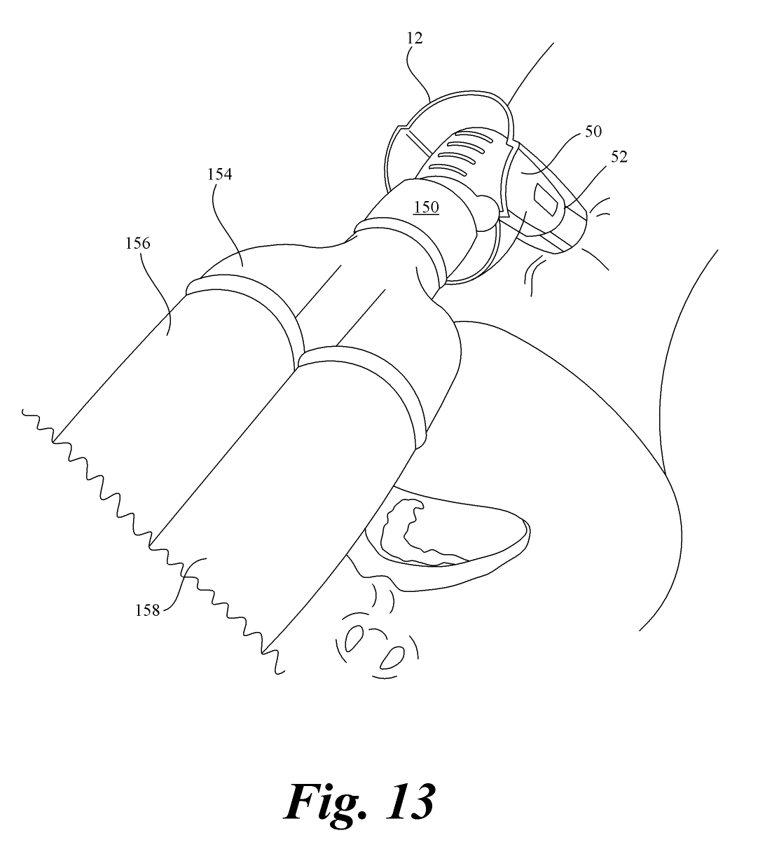

FIG. 1 schematically depicts a video-based cricothyrotomy ventilation system 10. System 10 includes a rigid sheath or cannula 12 having a blade region and a handle region, the blade region presenting a semi-sharp to substantially sharp V-like curvature to allow tissue separation. The blade region includes a curved region 14, a distal tip 16, a breathing aperture 17 located beneath the distal tip 16, a cuff 77 in pneumatic connection with an air port 45 via tubing 145, and a camera channel 142 (FIG. 4 below) that is pneumatically connected with the breathing aperture 17. The distal tip 16 may be spade-shaped to present a prying configuration that when used with the levering action conveyed through the curved region 14 facilitates tissue separation. The handle region includes a handle aperture 24, a securing tab 22, and a handle lumen 122 (FIG. 4 below) that is pneumatically connected with the camera channel 142. System 10 further includes an insertable and removable video camera 30, a ventilation adapter or pneumatic extension 50, and a monitor 100. The camera 30 includes a middle handle region 33 engageable with the handle lumen 122, a securing pin 36 slidably engageable with the tab 22, and extending from the proximal side of the camera handle 33 a video cable 38 that is in signal communication with the monitor 100 via connection port 115. The size, positioning, color rendition and contrast of any images presented on the monitor 100 may be adjusted by monitor controls 220. The ventilation extension or ventilation adapter 50 presents an angled tube configuration and includes a distal aperture 52 and a proximal aperture 54. Like the rigid cannula 12, the ventilation tube also has an anterior and a posterior side. The ventilation extension 50 has a securing pin 56 similar in function to the camera's 30 securing pin 36. The ventilation extension's 50 securing pin 56 similarly slidably engages the handle region's securing tab 22 to keep the ventilation extension 50 firmly in place during ventilation procedure and to allow it to be detachably removed from the cannula's 12 handle region. When the camera 30 is removed from the cannula's 12 channels 142 and 125, direct air communication is established between the distally located breathing aperture 17 and the handle aperture 24. When the ventilation adapter's 50 distal aperture 52 is sealably engaged against the handle lumen 122, a pneumatic continuation extends beyond the handle aperture 24 by the protrusion of ventilation adapter's 50 proximal aperture 54. Thus when the ventilation adapter 50 is sealably lodged or connected with the cannula's 12 handle region lumen 122, the protruded proximal aperture 54 is conveniently available for further pneumatic connection with a ventilation source, such as the ventilation tubes depicted in FIGS. 7 and 13 below.

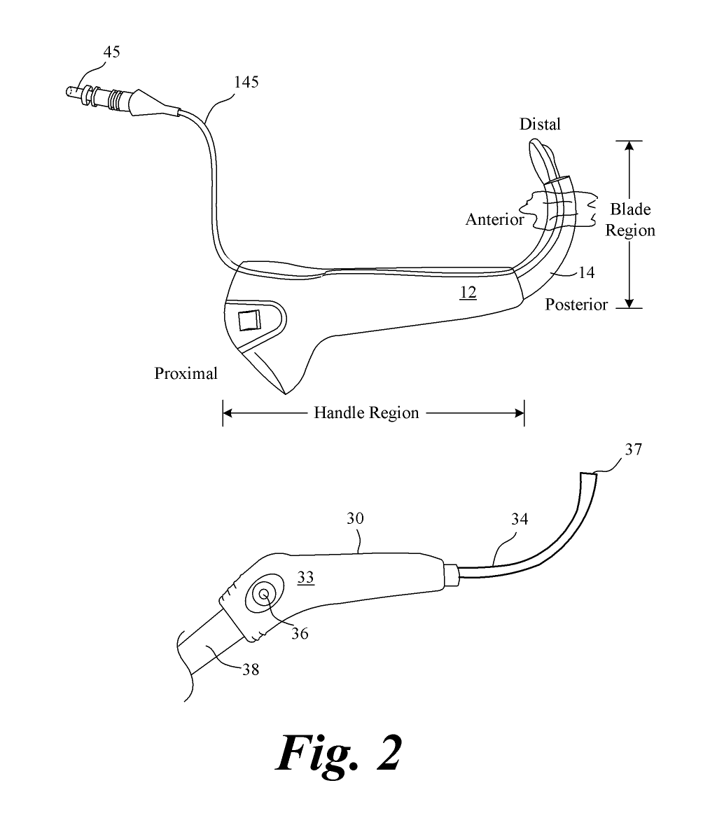

FIG. 2 schematically depicts the video camera 30 removed from the rigid cannula 12.

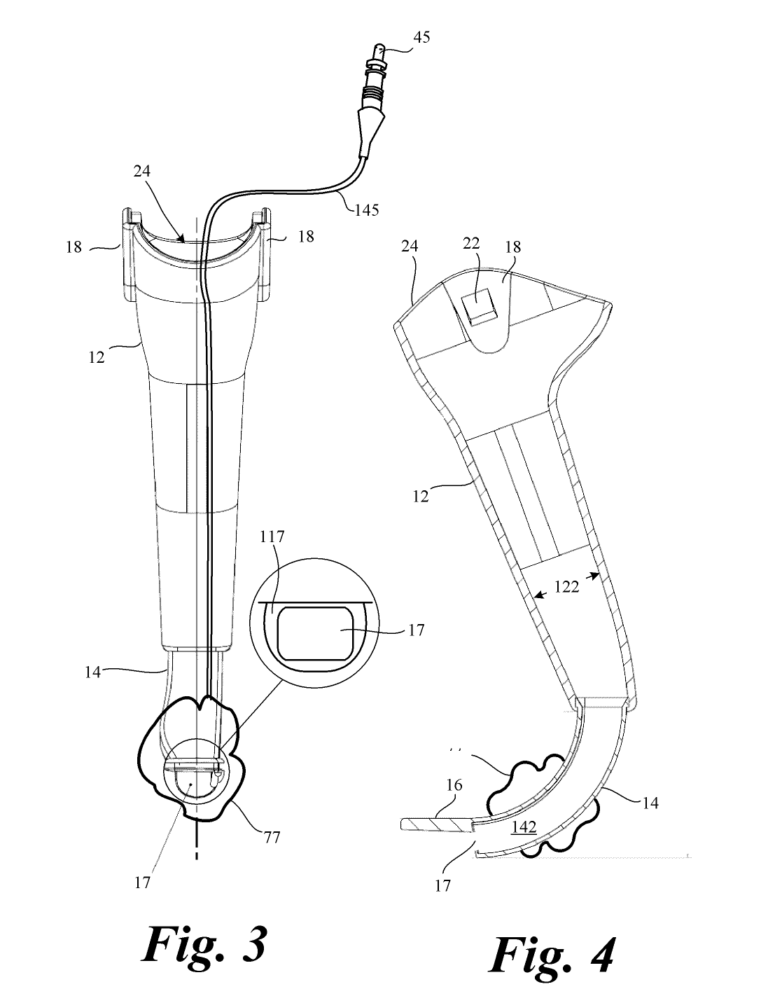

FIG. 3 schematically depicts an anterior view of the rigid cannula. Handle port 24 provides an aperture for inserting ventilation adapter 50 depicted in FIG. 1 above. Cuff 77 is in pneumatic communication airport 45 via tubing 145. Distal breathing port 17 is at the terminus of the blade region. Inset illustrates camera stops 117 that restrain the movement of the video camera beyond breathing port 17.

FIG. 4 schematically depicts a side cross-sectional view of the rigid cannula 12. Handle port or aperture 24 provides an aperture pneumatic communication of the handle region's lumen space 122 that extends from camera channel 142, and in turn to the breathing port 17. Thus when the camera 33 is removed from the cannula 12, an open air channel is established from the distal breathing port 17, through the camera channel 142, through the handle region lumen space 122, and then to the handle port 24.

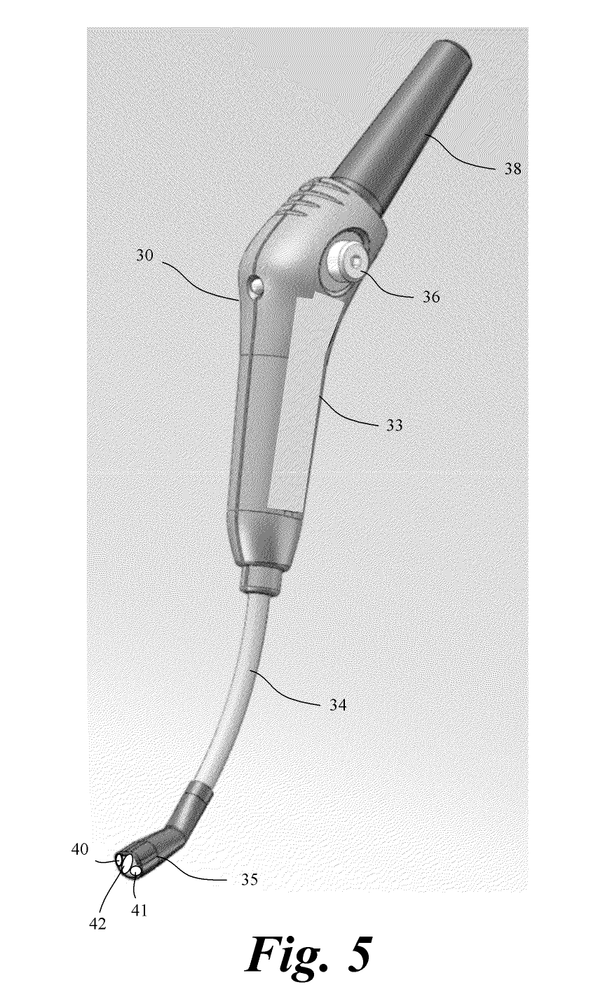

FIG. 5 schematically depicts a perspective view of the insertable and removable video camera 30. Camera face 35 includes a light source 41, camera lens 42, and a heated defogger port 40.

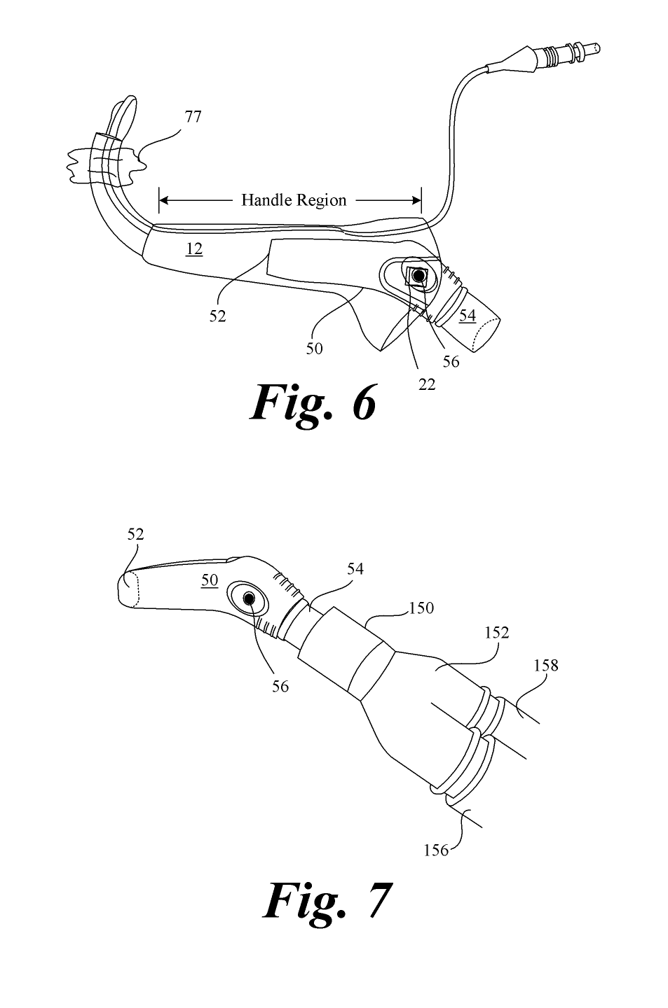

FIG. 6 schematically depicts a side view of the ventilation adapter 50 inserted into the handle portion of the rigid cannula 12. Distal aperture 52 of ventilation adapter 50 presses against the internal walls of the handle region at a location similar to handle lumen 122 depicted in FIG. 4 above. As depicted the ventilation extension 50 securing pin 56 in the process of sliding over the handle regions securing tab 22. Upon completion of sliding over to the distal side of the securing tab 22, the ventilation extension 50 is held firmly in place during ventilation procedures and may be detachably removed from the cannula's 12 handle region during non-ventilation procedures. Proximal aperture 54 of ventilation adapter 50 is available for sealable engagement with air portal 150 that is in pneumatic communication with ventilator tubes 156/158 via union 152 shown in FIG. 7 below.

FIG. 7 schematically depicts a perspective view of the attachment of the ventilation tubes 156/158 to the ventilation adapter 50. Proximal aperture 54 of ventilation adapter 50 is sealably engaged with air portal 150 that is in pneumatic communication with ventilator tubes 156/158 via union 152.

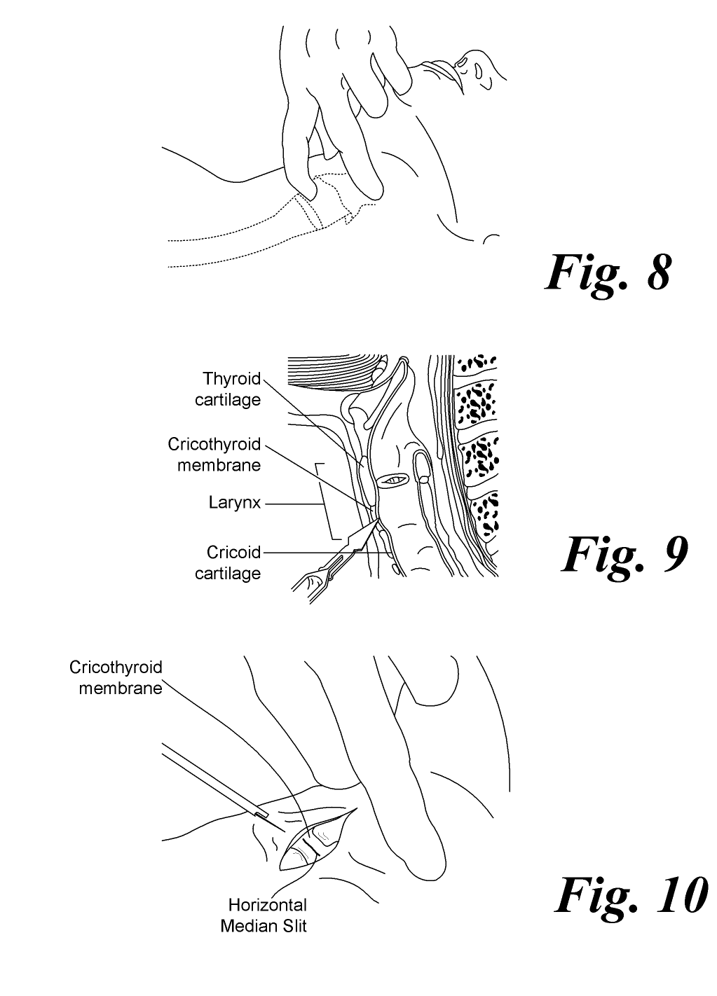

FIGS. 8-10 depict the anatomical location and preparation of the cricrothyroid membrane for receiving entry of the rigid cannula.

In FIG. 8, the larynx is digitally located.

FIG. 9 illustrates in cross section the location for preparing a substantially horizontal cut through the cricothryorid membrane of the larynx.

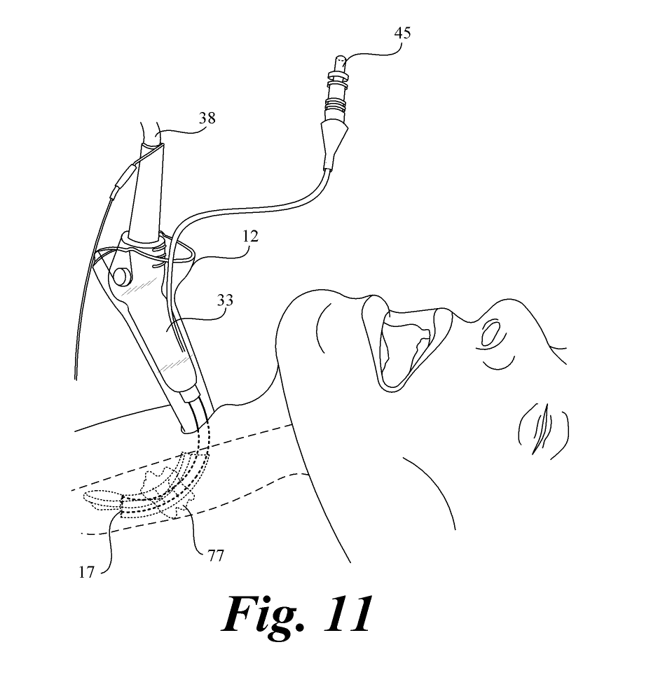

FIG. 10 schematically depicts a substantially lateral or horizontal median slit made into the cricothyroid membrane after a substantially horizontal cut is made along the patient's superior-inferior median line in the neck region to expose the cricothyroid membrane.

FIG. 11 schematically depicts the insertion of the camera-loaded cannula's 12 blade region into the tracheal lumen with the video camera lens 45 (as depicted in FIG. 5 and elsewhere above) viewing inferiorly towards the lungs of the patient. The breathing aperture 17 is similarly aimed inferiorly towards the lungs of the patient. Cuff 77 is not yet inflated to secure anchoring of the blade region of the cannula 12 against the tracheal walls.

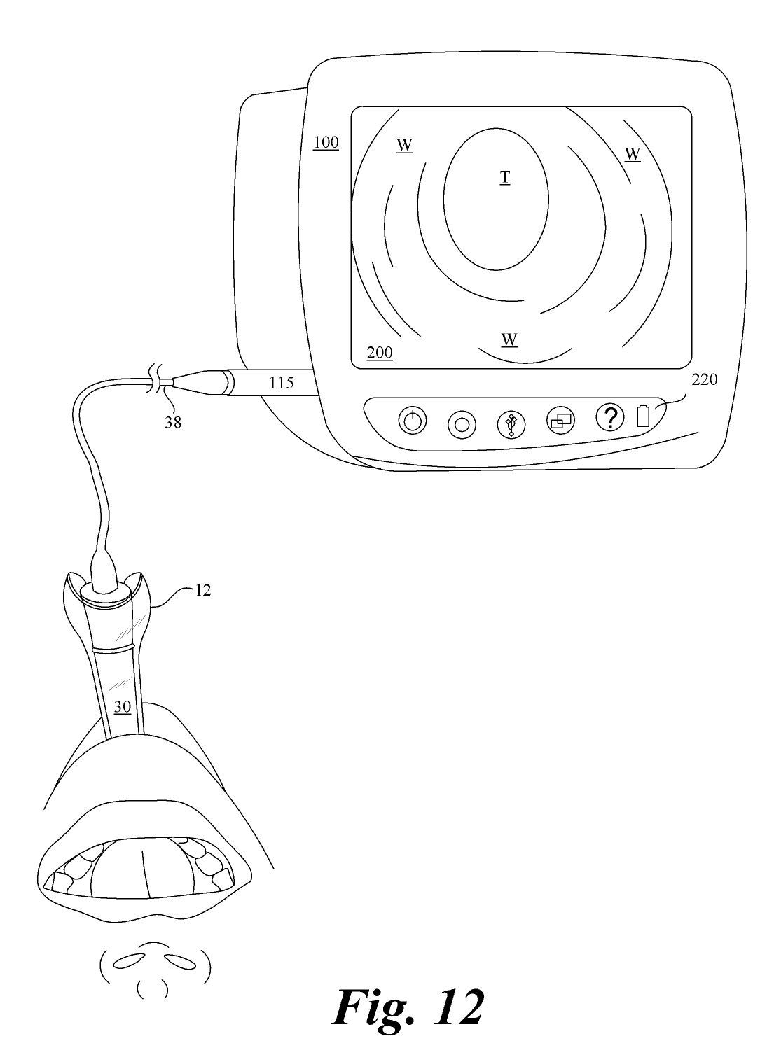

FIG. 12 schematically depicts the camera-loaded cannula's 12 handle region lodged in and projecting from the patient's neck in which the video camera 30 provides an interior view of the tracheal lumen presented on the monitor 100 showing a substantially centered blade region as indicated by the walls W defining the tracheal lumen T appearing in the middle of the monitor image. The tracheal lumen T is shown substantially in the middle of the video image 200 conveyed by the camera lens 45 of video camera 30 (as depicted in FIG. 5 and elsewhere above) presented on the monitor 100. The size, positioning, color rendition and contrast of the image 200 may be adjusted by monitor controls 220.

FIG. 13 schematically depicts a perspective view of the attached ventilator tubes 156/158 pneumatically in communication with the ventilator adapter 50 snugly held within the cannula's 12 handle region that protrudes from the patient's neck. Tubes 156/158 through union 154 are pneumatically connected via port 150 that slidably overlaps adapter's 50 proximal port 52 (not shown). The adapter's 50 distal port 52 snugly fits or sealably engages the internal walls defining the lumen space 122 of the cannula's 12 handle region depicted in FIG. 4 above.

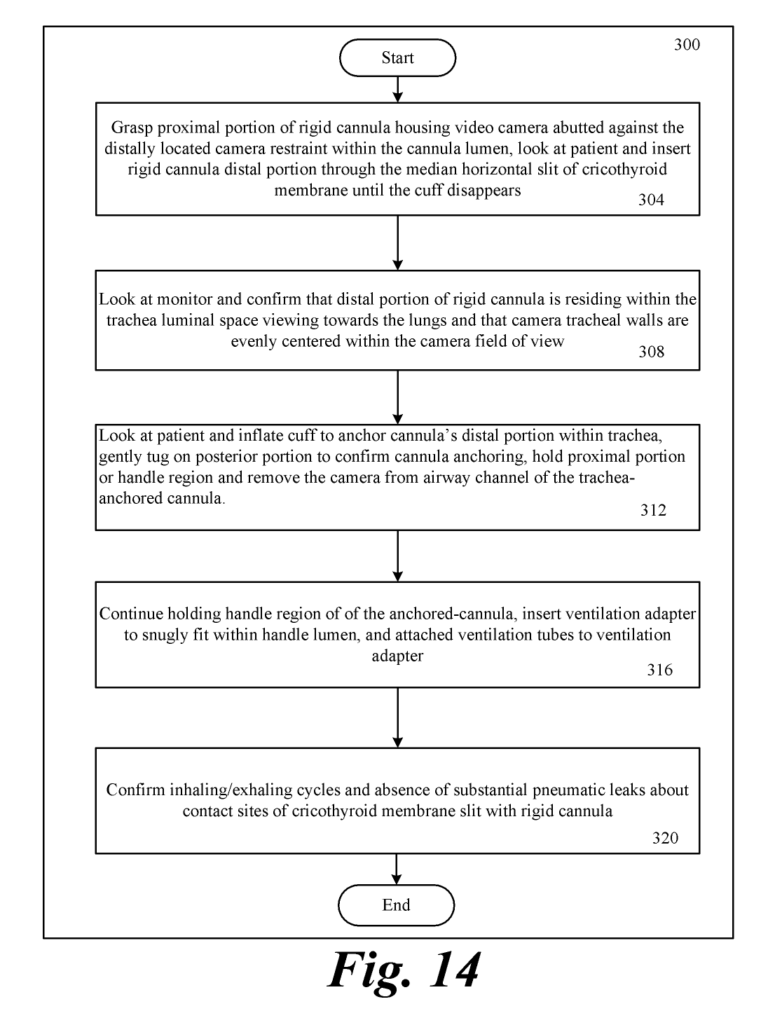

FIG. 14 schematically depicts a method 300 to of using the video-based cricothyrotomy ventilation system 10 to secure a breathing communication with the patient's airway. With reference to FIGS. 1-13 above, beginning at process block 304, the proximal portion of the rigid cannula 12, the handle region, is grasped by the user who confirms the video camera 30 is already inserted and securely lodged in the lumen of the handle region such that the lens, light source, and antifogging port of the video camera face is in contact with camera stop 117 circumscribing the airway port 17 shown in FIGS. 3, 4, and elsewhere above. The user looks at the patient's cricothyroid membrane horizontal slit and inserts the tip 16 into the slit such that its entry into the tracheal lumen will be pointed inferiorly towards the patient's lungs and away from the patient's head. The user continues with the insertion until the cuff 77 disappears from the user's view. Thereafter, at block 308, the user looks at the monitor 100 to confirm that the distal portion or the blade region of the rigid cannula 12 has a good, centered view of the trachea T in that the tracheal walls circumscribing the blade region are substantially evenly spaced within the camera field of view as shown in video images presented on the monitor 100. The user manipulates the handle region of the cannula 12 until a centered view of the trachea T is obtained. Thereafter, at process block 312, the user looks at the patient's neck region and inflates the cuff 77, conveniently via a syringe loaded with an air bolus connected to air port 45. The cuff expands and substantially presses evenly against the tracheal walls to anchor the blade region of the cannula 12 against the surrounding tracheal walls. While looking at the handle region of the cannula 12, the user confirms in-trachea anchoring of the blade region of the cannula 12 by tugging gently the handle region to confirm that the cannula 12 does not slip out of the cricothyroid membrane horizontal slit. Thereafter, at process block 316, the video camera 30 is removed from the in-trachea anchored cannula 12 allowing pneumatic communication through the handle region's lumen to the blade region's ventilation aperture 37. The ventilation adapter's 50 distal aperture 52 is inserted to snugly fit into the lumen space of the handle region of cannula 12, then the ventilation tube assembly 150 to the ventilation adapter's 50 proximal aperture 52. Alternatively, the assembly 150 may be snugly press fitted with the proximal opening 54 of ventilator adapter 50, and then the ventilator adapter's 50 distal aperture press fitted inside the lumen space of the cannula's 12 handle region. Then, adequate ventilation communication is confirmed at process block 320 wherein the absence of substantial leaking along the contact regions of the rigid cannula 12 is confirmed during the patient's breathing exhibited during inhalation and exhalation cycles.

While the preferred embodiment of the invention has been illustrated and described, as noted above, many changes can be made without departing from the spirit and scope of the invention. For example, in alternate embodiments the substantially rigid cannula 12 may be configured to include a flexible fiber optic or video scope having a partially flexible or controllable tip 16 to facilitate migration and placement of the cannula within the tracheal lumen. Similarly, monitor 100 may be in signal communication with, and wirelessly coupled to, the video camera 30 via Bluetooth® or other wireless communication technologies. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A cricothyrotomy system for establishing an airway with a ventilation source via the tracheal lumen exposed by a slit placed in the cricothyroid membrane, the system comprising:
a substantially rigid cannula having a channel with a proximal and distal port configured for entry into the slit;
a camera slidable through the channel to reach the distal port;
a monitor in signal communication with the camera; and
an extension sealably engageable with the channel and the ventilating source,
wherein positioning is determined by images conveyed by the camera and presented on the monitor.

2. The cricothyrotomy system of claim 1, wherein the inflatable cuff is expanded upon appearance of images showing a centered tracheal lumen.

3. The cricothyrotomy system of claim 1, wherein replacement of the camera with the extension provides pneumatic connection between the distal port located within the tracheal lumen and ventilation source.
